(19) United States
(12) Patent Application Publication
(10) Pub. No.: US 2008/0050779 A1
(43) Pub. Date: Feb. 28, 2008

(54) Fermentation Process Using Faba Beans as Nitrogen Source

(76) Inventors: Aurelia Defachelles, Lomme (FR); Vincent Jean-Pierre Christian Dubois, Lambesart (FR); Philippe Thierry Francois Gaudin, Den Hoorn (NL)

Correspondence Address: NIXON & VANDERHYE, PC, 901 NORTH GLEBE ROAD, 11TH FLOOR, ARLINGTON, VA, 22203, US

(21) Appl. No.: 11/629,201

(22) PCT Filed: Jun. 15, 2005
(86) PCT No.: PCT/EP05/52761

§ 371 Date: Jan. 11, 2007

(30) Foreign Application Priority Data
Jun. 16, 2004 (EP) 04076774.1

(51) Int. Cl. C12P 1/00

(52) U.S. Cl. 435/71.1; 435/41

(57) ABSTRACT

The present invention describes a microbial fermentation process using a fermentation medium comprising a complex nitrogen source, wherein a substantial part of the nitrogen (N) that is supplied by the complex nitrogen source is provided by faba bean meal. The use of faba bean meal has a viscosity lowering effect as compared to the use of a complex nitrogen source other than faba bean meal.

The present invention relates to the field of microbial fermentation.

In microbial fermentation processes, a microorganism typically is fermented in a fermentation medium comprising a complex nitrogen and/or carbon source. Examples of complex nitrogen and/or carbon sources include soybean meal, cottonseed meal, corn steep liquor, yeast extract, casein hydrolysate, molasses, and the like. The composition of fermentation media, in particular the composition of the complex nitrogen and/or carbon source, may have an important influence on fermentation parameters like viscosity, heat transfer and oxygen and nutrient transfer. In that regard, a low viscosity of the fermentation broth is advantageous for biomass as well as product formation and therefore control of viscosity is of the utmost importance in industrial scale fermentation processes.

WO 02/26961 discloses a process for producing surfactin by cultivating microoranisms of the genus Bacillus in a culture medium comprising flour of beans, in particular selected form the group consisting of soybean, adzuki bean, pea, broad bean, chick pea, lentil and string bean, or yeast extract, whereby soybean is preferred among the beans. As an example, fermentation in a 5 L fermenter is shown using a medium comprising soybean and yeast extract.

Abdel-Hafez (Naturalia monspeliensia 50, 1986, 109-118) describes that the filamentous fungi count of soil enriched with among other broad bean powder as carbon source and sodium nitrate as nitrogen sources is influenced by the C/N ratio of the added carbon and nitrogen sources.

The documents mentioned above do not teach the advantageous use of faba bean meal in industrial scale fermentation processes. The present invention surprisingly shows that the use of faba bean meal in fermentation media of industrial scale fermentation processes provides a substantial decrease in viscosity of the fermentation broth as compared to the use of other complex nitrogen sources such as soybean meal.

The present invention discloses a process for the preparation of microbial biomass and/or a valuable compound derived form microbial fermentation comprising fermentation of a microorganism in a medium comprising a complex nitrogen source, characterised in that a substantial part of the nitrogen (N) that is supplied by the complex nitrogen source is provided by faba bean meal.

Thus, in a fermentation process according to the invention, faba bean meal provides a substantial part of the nitrogen (N) that is supplied as a complex nitrogen source, i.e. that is introduced into the fermentation process as complex nitrogen source. In this context, a “substantial part” of the nitrogen (N) is intended to mean at least 50% of the nitrogen (N), preferably at least 60%, more preferably at least 70%, more preferably at least 80%, most preferably at least 90%. In particular, all complex nitrogen source that is introduced into the fermentation process is provided by faba bean meal. Nitrogen (N) thereby is conveniently expressed as Kjeldahl nitrogen.

It is envisaged by the present invention that an amount of complex nitrogen source other than faba bean meal may be present in the process of the invention, for instance as carry-over from the inoculum for the main fermentation. Typical examples of complex nitrogen sources other than faba bean meal are soybean meal, yeast extract, cottonseed meal, corn steep liquor, casein hydrolysate. Preferably, the complex nitrogen source other than faba bean meal is yeast extract.

In the fermentation process according to the invention, additional nitrogen than that provided by a complex nitrogen source may be introduced or present in the fermentation process. Typically, such additional nitrogen is provided by a chemically defined nitrogen source, like ammonia, ammonium salts or nitrate salts. For instance, ammonia may be used for pH regulation during the whole course of the fermentation process or during a particular time period.

The term “faba bean meal” as used in the context of the present invention is intended to encompass meal or flour obtainable from faba beans by pulverising faba beans and, optionally, supplementing with an aqueous extract and/or a hydrolysate (acid or enzymatic) of (pulverised) faba beans. The aqueous extract and/or hydrolysate may comprise 0-100% of the faba bean meal. Faba beans (also called broad beans) include beans obtainable from Vicia faba and/or from related varieties or species.

Other components of the fermentation medium necessary for performing the fermentation process according to the invention are not critical to the invention, but are added to the process according to the need of the organism in question.

In addition to the nitrogen source, the fermentation medium conveniently contains a carbon source as well as additional compounds required for growth of the microorganism and/or the formation of certain valuable compounds. For instance, additional compounds may be necessary for inducing the production of a valuable compound.

Examples of suitable carbon sources known in the art include glucose, maltose, maltodextrins, sucrose, hydrolysed starch, starch, molasses, oils. Examples of additional compounds include phosphate, sulphate, trace elements and/or vitamins.

The total amount of carbon and nitrogen source to be added to the fermentation process according to the invention may vary depending on e.g. the needs of the microorganism and/or the length of the fermentation process.

The ratio between carbon and nitrogen source in a fermentation process may vary considerably, whereby one determinant for an optimal ratio between carbon and nitrogen source is the elemental composition of the product to be formed.

Additional compounds required for growth of a microorganism and/or for product formation, like phosphate, sulphate or trace elements, may be added in amounts that may vary between different classes of microorganisms, i.e. between fungi, yeasts and bacteria. In addition, the amount of additional compound to be added may be determined by the type of valuable compound that is formed.

Typically, the amount of medium components necessary for growth of a microorganism may be determined in relation to the amount of carbon source used in the fermentation, since the amount of biomass formed will be primarily determined by the amount of carbon source used

The fermentation process according to the invention is preferably performed on an industrial scale. An industrial scale process is understood to encompass a fermentation process on a fermenter volume scale which is ≧0.01 m³, preferably ≧0.1 m³, preferably ≧0.5 m³, preferably ≧5 m³, preferably ≧10 m³, more preferably ≧25 m³, most preferably ≧50 m³.

Any microorganism that produces a valuable compound or that is to be used as such as biomass may be subjected to the fermentation process according to the invention, e.g. bacterial, yeast and fungal organisms. For instance, microbial strains which are suitable for fermentation according to the invention include fungal strains, such as Aspergillus, Penicillium or Mucorales strains, yeast strains, such as Saccharomyces, Pichia, Phaffia or Kluyveromyces strains and bacterial strains, such as Bacillus or Actinomycete strains. Especially filamentous organisms will benefit from the use of faba beans in a fermentation process according to the invention. Filamentous organisms can be filamentous bacteria, like Actinomycetes, preferably Streptomyces, or filamentous fungi. Preferably, the filamentous fungus originates from the order Mucorales or the genus Acremonium, Aspergillus, Fusadium, Penicillium, Rhizomucor, Rhizopus, Talaromyces, Trichoderma,

The microbial strain to be subjected to the process of the invention may be a naturally occurring microorganism, or a microbial strain derived from any suitable parent strain by any kind of mutagenesis technology, e.g. classical mutagenesis treatment or genetic engineering technology.

The valuable compound produced by the microorganism to be subjected to the process of the invention may be a protein, e.g. an enzyme or a pharmaceutical protein, or a primary or secondary metabolite, e.g. an antibiotic, carotenoid or vitamin.

Suitable enzymes to be produced according to the invention are hydrolases and oxidoreductases. Hydrolases include proteases, peptidases, amylases, phosphatases, phytases, cellulases, hemicellulases, glucanases, lipases.

A fermentation process according to the invention can be performed as a batch, a repeated batch, a fed-batch, a repeated fed-batch or a continuous fermentation process.

In a batch process, all medium components are added directly, as a whole, to the medium before the start of the fermentation process.

In a repeated batch process, a partial harvest of the broth accompanied by a partial supplementation of complete medium occurs, optionally repeated several times.

In a fed-batch process, part of the compounds necessary for microbial growth and/or product formation is supplied in the starting medium, prior to starting the fermentation process. In the course of the fermentation process, additional carbon source, nitrogen source and/or additional compounds as desired may be fed to the process, in one feed or in a separate feed for each compound.

In a repeated fed-batch or a continuous fermentation process, the complete starting medium is additionally fed during fermentation. The starting medium can be fed as a whole or in separate streams of e.g. carbon and nitrogen source. In a repeated fed-batch process, part of the fermentation broth comprising the biomass is removed at regular time intervals, whereas in a continuous process, the removal of part of the fermentation broth occurs continuously. The fermentation process is thereby replenished with a portion of fresh medium corresponding to the amount of withdrawn fermentation broth.

In a preferred embodiment of the invention, a fed-batch or repeated fed-batch process is applied, wherein the carbon source and/or the nitrogen source and/or additional compounds are fed to the fermentation process. In a more preferred embodiment, the carbon and/or nitrogen source are fed to the fermentation process.

In another preferred embodiment of the invention, a controlled feed is applied in the fermentation process in such a way to effectuate a process under carbon-limited conditions. Optionally, the feed also contains additional nitrogen and/or salts to avoid other limitation than carbon.

In another preferred embodiment of the invention, part of the complex nitrogen source, a substantial part of which is faba bean meal, is supplied in the starting medium, whereas additional complex nitrogen source is fed in the course of the fermentation process. The nitrogen (N) provided by the complex nitrogen source supplied in the feed, i.e. fed to the process in the course of fermentation, may preferably be up to 80% inclusive of the total amount of complex nitrogen used, preferably up to 70% inclusive, preferably up to 60% inclusive. Additional nitrogen added may be in chemically defined form, e.g. in the form of ammonia for pH control.

Faba bean meal is typically applied in the starting medium in a concentration that may range from 10 to 70 g/L. The concentration of faba bean meal in the feed may range from 5 to 30 g/L.

The use of a fed-batch process typically enables the use of a considerably higher amount of carbon and nitrogen source than is used in a batch process. Specifically, the amount of carbon and nitrogen source applied in a fed-batch process can be at least about two times higher than the highest amount applied in a batch process. This, in turn, leads to a considerably higher amount of biomass formed in a fed-batch process.

The present invention surprisingly shows that the use of faba bean meal provides an increase in the productivity of a compound of interest, especially an increase in enzyme productivity.

A further aspect of the present invention concerns the option of downstream processing of the fermentation broth. The use of faba bean meal according to the invention facilitates downstream processing, especially because of the relatively low viscosity of the fermentation broth. Downstream processing may include recovery as well as formulation steps.

After the fermentation process is ended, the valuable product may be recovered from the fermentation broth, using standard technology developed for recovery of the valuable compound of interest. The relevant downstream processing technology to be applied thereby depends on the nature and cellular localization of the valuable compound and on the desired purity level of the product of interest. In a typical recovery process, the biomass is separated from the fermentation fluid using e.g. centrifugation or filtration. The valuable compound then is recovered from the biomass in the case that the valuable product is accumulated inside or is associated with the microbial cells. Of course, the biomass as such may also be used. Otherwise, when the valuable product is excreted from the microbial cell, it is recovered from the fermentation fluid.

The biomass and/or valuable compound may be formulated as liquid or solid products.

### EXPERIMENTAL

### DESCRIPTION OF THE FIGURES

FIG. 1: Bacillus amyloliquefaciens. Graphs represent dissolved oxygen (% of air saturation, close symbols) and Oxygen Uptake Rate (mmol/l/h, open symbols). Circle: Reference; triangle: Test. X-axis: time after inoculation (h).

FIG. 2: Bacillus amyloliquefaciens, protease activity evolution. All data are normalised by the highest activity measured for Reference experiment. Circle: Reference; triangle: Test.

FIG. 3: Rhizomucor miehei, viscosity evolution versus time. Square: Reference; triangle: Test.

FIG. 4: Rhizomucor miehei, productivity versus time.

### GENERAL METHODS

Protease Assay

1 PCU protease corresponds to enzymatic activity that produces in one minute a hydrolysate with the same 275 nm optical density as a 1.5 μg/ml Tyrosine solution.

The enzyme was allowed to react upon a casein solution for 30 minutes at 37° C. and at pH 7.0. After stopping the reaction with TCA solution and filtration, the absorbance of the clear top-layer was spectrophotometrically measured at 275 nm.

Daily prepared substrate is Casein Hammarsten Merck (7 g/L in 0.05 M Tris and 0.016 M HCl, dissolved by heating for 30 minutes in a boiling water bath, cooled down to room temperature, pH adjusted to 7.0±0.02 (HCl 0.2 M), volume completed to 1 L with demi water, and solution filtered on Whatman filter n^{o}42). Buffer is 0.433 g/L CaCl₂, 2 H₂O, 0.14 g/L MgCl₂, 6 H₂O, 0.21 g/L anhydrous NaHCO₃, 12.1 g/L Tris, pH 7±0.02 (HCl 1M). This buffer solution is used to prepare enzymatic solutions of 11-25 units/ml. TCA solution is 18 g/L trichloric acetic acid, 19 g/L sodium acetate, 3 H₂O and 20 g/L acetic acid.

Assay is as follows: 10 ml of substrate solution into plugged tubes, in a water bath at 37° C. for 10 minutes; at time 0, add 2 ml of enzymatic solution; mix thoroughly and allow to react for 30 minutes exactly; add 10 ml of TCA solution; mix thoroughly and leave at 37° C. for 30 minutes; mix and filtrate on Whatman paper n^{o}42. The filtrate optical density is measured in quartz cuvette of 10 mm optical trajectory, and corrected for a blank (incubated casein solution, after 30 minutes first add TCA solution, immediately followed by 2 ml sample solution). The result is compared to a calibration curve obtained by optical density of Tyrosine 25, 40, 75 μg/ml (stock solution 100 μg/ml, with first dissolution of 100 mg L(−) Tyrosine into 60 ml HCl 0.1M).

Viscosity Assay

Viscosity was measured according to the “Epprecht method” with the TVe-05 equipment from LAMY. Analysis procedure is according to the one described in the supplier manual. Attention should be paid that analysis is realized in a short period after sampling (less than 30 min).

### EXAMPLE 1

### Bacillus amyloliquefaciens

A recombinant strain of Bacillus amyloliquefaciens producing protease was used. For pre-culture, one deep-frozen culture (1 ml vial) was used to inoculate 200 mL of a medium prepared as follows: glucose 2.5 g/l, Yeast Extract Biospringer Powder 20 g/L, K₂HPO₄ 2.5 g/L, antifoam (Clérol FBA 3107) 0.6 g/L, pH set to 7.0 with NaOH 4N, sterilisation.

After pre-culture (shake flask, 10 h, 250 rpm, 34° C.) a 10 L main fermentor was inoculated. The working volume was 6 L, with the following medium composition: Soy bean meal (Nutrisoy, ADM) 66.6 g/L, spay-dried corn steep liquor (Solulys A ST, Roquette) 13.3 g/L, (NH₄)₂HPP₄ 6.6 g/L, CaCl₂.2 H₂O 1.3 g/L, NaCl 0.66 g/L, ZnSO₄.7 H₂O 0.066 g/L, MnSO₄.1H₂O 0.06 g/L, Clérol FBA 3107 2 ml/L, glucose.1 H₂O 50 g/L (sterilised apart). pH is corrected to 6.1±0.1 (NaOH) for the salt fraction prior to sterilisation. For Test fermentation, soy bean meal was replaced (same concentration) by faba bean meal (CPX55, GEMEF Industries). After inoculation, the main fermentation was run at 37° C., pH 5.9 (using NH₄OH or H₃PO₄), airflow 5 normal-L/min, agitation rate between 300 and 700 rpm being controlled by a dissolved oxygen level set-point at 35% of oxygen saturation by air. Antifoam was added at frequency of 2 sec. every 15 min. (2 sec. every 7 min. between 10 and 20 h after inoculation). A feed of glucose (131 g/h, feed at 470 g/L in glucose.1 H₂O) was started once oxygen uptake rate had reached 10 mmol/L/h. The whole main fermentation lasted 30 h.

Samples were regularly taken (same amount for each reactor at every sampling time), and supernatant was analysed for protease activity (PCU/ml).

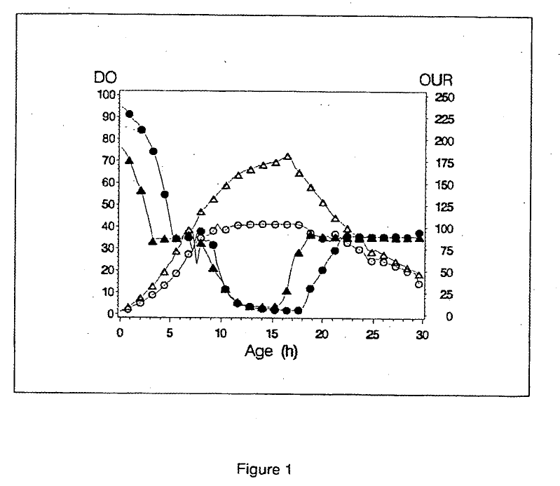

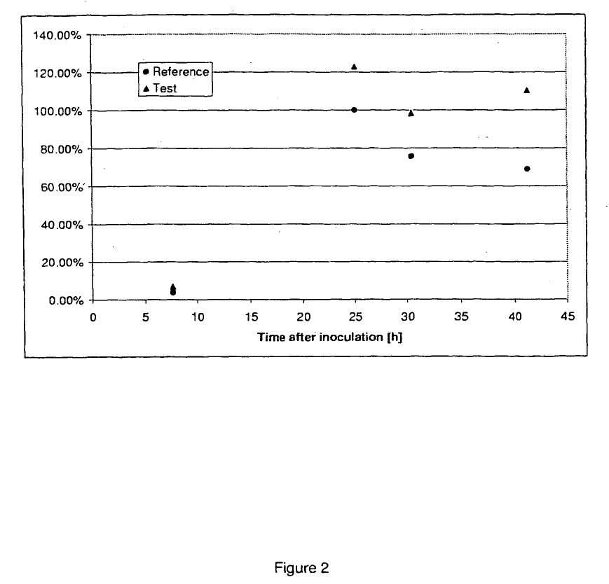

As can be seen in FIG. 1, experiments with Faba Bean led to a better oxygen transfer through a lower broth viscosity. FIG. 2 shows clearly the advantage brought about by faba bean meal for the productivity.

### EXAMPLE 2

### Rhizomucor miehei

A strain of Rhizomucor miehei producing protease was used. For pre-culture, one deep-frozen culture (1 ml per vial) was used to inoculate 100 mL of a medium prepared as follows: dextrose 20 g/L, yeast extract Gistex liquid 20 g/L, K₂HPO₄ 1 g/L, Tween 80 2 g/L, pH set to 6.0 with NaOH 5N, sterilisation.

After pre-culture (shake flask, 30 h, 250 rpm, 39° C.) a 10 L main fermenter was inoculated. The working volume was 4 L initially, with the following medium composition: Soy bean meal (Nutrisoy, ADM) 50 g/L, (NH₄)₂SO₄ 2 g/L, antifoam (Clérol FBA 3107) 0.23 g/L, dextrose 44 g/L, KH₂PO₄ 4 g/L, CaCl₂.2H₂O 0.1 g/L, citric acid 1 g/L, MnSO₄.H₂O 0.01 g/L, MgSO₄.7 H₂O 1 g/L, FeSO₄.7H₂O 0.1 g/L and ZnSO₄.7H₂O 0.01 g/L. Nutrisoy, (NH₄)₂SO₄ and antifoam (Clérol FBA 3107) were separately sterilized. pH is corrected to 6.5±0.1 (NaOH) for the nitrogen fraction prior to sterilisation and 3.5±0.1 (H₃PO₄) for carbon and salt fraction. For Test fermentation, soy bean meal was replaced (same concentration) by faba bean meal (CPX55, GEMEF Industries). After inoculation, the main fermentation was run at 39° C., pH 5.4 (using NH₄OH or H₃PO₄), airflow 8 normal-L/min, agitation rate between 700 and 900 rpm according to a defined profile. A complete feed was prepared as follow: Nutrisoy 17 g/L, Clérol 0.1 g/L, dextrose 85 g/L, KH₂PO₄ 5 g/L, CaCl₂.2H₂O 0.1 g/L, citric acid 1 g/L, MnSO₄.H₂O 0.01 g/L, MgSO₄.7 H₂O 1 g/L, FeSO₄.7H₂O 0.1 g/L and ZnSO₄.7H₂O 0.01 g/L. Nutrisoy was separately sterilized and fraction pH was adjusted to 4.50±0.1 before sterilization. Feed was started once glucose depletion in batch was observed according to a determined profile. The whole main fermentation lasted 120 h.

Samples were regularly taken (same amount for each reactor at every sampling time), and supernatant was analysed for protease content.

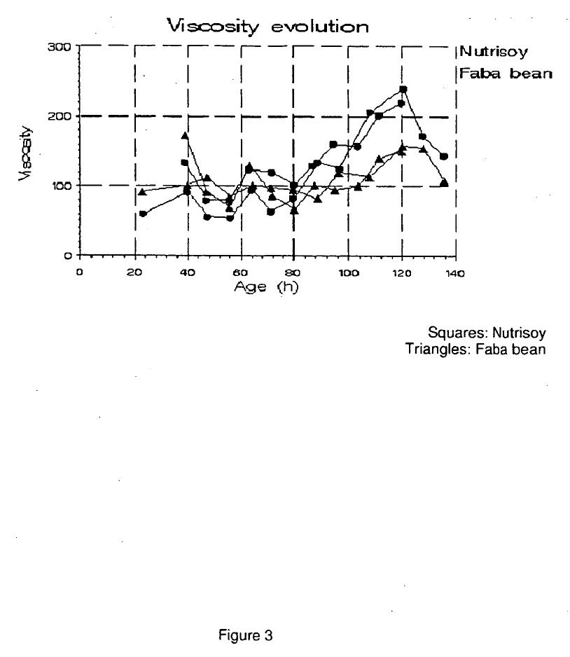

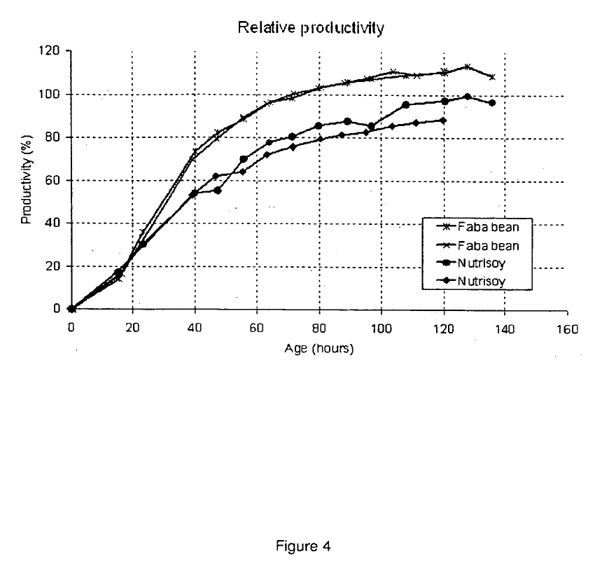

As can be seen in FIG. 3, experiments with faba bean meal led to a lower viscosity development in fermentation broth after a certain time. FIG. 4 shows clearly the advantage brought about by faba bean meal for the productivity.

1. A process for the preparation of microbial biomass and/or a valuable compound comprising fermentation of a microorganism in a medium comprising a complex nitrogen source, characterised in that a substantial part of the nitrogen (N) that is supplied by the complex nitrogen source is provided by faba bean meal.

2. A process according to claim 1, wherein a substantial part of the nitrogen (N) is at least 50% of the nitrogen (N), preferably at least 60%, more preferably at least 70%, more preferably at least 80%, most preferably at least 90%.

3. A process according to claim 1, wherein the complex nitrogen source further comprises yeast extract.

4. A process according to claim 1, wherein the fermentation is performed on an industrial scale.

5. A process according to claim 1, wherein the valuable compound is a protein or a primary or secondary metabolite.

6. Use of faba bean meal in microbial fermentation to decrease the viscosity of the fermentation broth as compared to using a complex nitrogen source other than faba bean meal.
